(19) United States
(12) Patent Application Publication
(10) Pub. No.: US 2003/0171765 A1
(43) Pub. Date: Sep. 11, 2003

(54) Catheter for treating vulnerable plaque

(75) Inventors: Jaydeep Y. Kokate, Maple Grove, MN (US); Eric M. DoBrava, Crystal, MN (US)

Correspondence Address: FISH & RICHARDSON P.C., 3300 DAIN RAUSCHER PLAZA, 60 SOUTH SIXTH STREET, MINNEAPOLIS, MN, 55402, US

(73) Assignee: SciMed Life Systems, Inc.

(21) Appl. No.: 10/091,871

(22) Filed: Mar. 5, 2002

(51) Int. Cl. A61B 17/22

(52) U.S. Cl. 606/159; 606/194

(57) ABSTRACT

Devices and methods for removing core material contained in plaque deposits inside a blood vessel are disclosed. A device in accordance with one embodiment of the present invention includes a catheter consisting of an elongate shaft, a balloon disposed about a portion of the elongate shaft for urging the core material from the plaque deposits, and a plurality of collection lumens with distal ends disposed proximally and/or distally of the balloon for extracting the urged core material. A method in accordance with one embodiment includes the steps of inserting a distal portion of the catheter into a lumen of the blood vessel, positioning the balloon proximate a plaque deposit, inflating the balloon to rupture the plaque and urge the core material therefrom, and extracting the urged core material by suction means at the proximal ends of the collection lumens.

### FIELD OF THE INVENTION

The present invention relates generally to intravascular catheters. More particularly, the present invention relates to intravascular catheters adapted to remove vulnerable plaque.

### BACKGROUND OF THE INVENTION

Atherosclerotic coronary artery disease is a leading cause of death in industrialized countries. An atherosclerotic plaque is a thickening in the wall of the artery. Typically, patients who have died of coronary disease may have several atherosclerotic plaques; however, in most instances of myocardial infarction, cardiac arrest, or stroke, it is found that only one of these potential obstructions has, in fact, ruptured, fissured, or ulcerated. The rupture, fissure, or ulcer typically causes a large thrombus to form on the inside of the artery, which may completely occlude the flow of blood through the artery, thereby injuring the heart and/or the brain.

Plaque, a thickening in the arterial vessel wall results from the accumulation of cholesterol, proliferation of smooth muscle cells, secretion of a collagenous extracellular matrix by the cells, and accumulation of macrophages and eventually, hemorrhage (bleeding), thrombosis (clotting), and calcification. The consensus theory is that atherosclerotic plaque develops as a result of irritation or biochemical damage to the endothelial cells which line the inner surface of the blood vessel. Endothelial cells normally prevent inappropriate formation of blood clots and inhibit contraction and proliferation of the underlying smooth muscle cells. Most investigators believe that atherosclerotic plaques can develop when endothelial cells are damaged or dysfunctional. Dysfunctional endothelial cells typically result from cigarette smoking, high serum cholesterol (especially oxidized low density lipoprotein), hemodynamic alterations (such as those found at vessel branch points), some viruses (herpessimplex, cytomegalovirus) or bacteria (e.g., chlamydia), hypertension, some hormonal factors in the plasma, and other factors as yet unknown. As a result of these gradual injuries to the endothelial cells, an atherosclerotic plaque may grow slowly over many years.

When a plaque rupture develops, there is typically a hemorrhage into the plaque through a fissure where the surface of the plaque meets the bloodstream. A thrombus quickly forms upon contact with the collagen and lipid of the plaque. This blood clot may then grow to completely occlude the vessel, or it may remain only partly occlusive. In the latter case, the new clot quite commonly becomes incorporated into the wall of the plaque, creating a larger plaque.

The condition of plaque deposits can vary. For example, the plaque can be inflamed and unstable, or the plaque can be quite stable. Plaque deposits that are at risk of rupturing are sometimes referred to as vulnerable plaque. Vulnerable plaque typically include a core of soft material covered with a fibrous cap. Many of vulnerable plaque deposits do not limit the flow of blood through the blood vessels. It has recently been appreciated that vulnerable plaques that do not limit flow may be particularly dangerous because they produce no warning symptoms, and can suddenly rupture causing heart attack, stroke, and/or death by forming a blood clot inside the blood vessel lumen and causing a blockage, for example.

### SUMMARY OF THE INVENTION

A device in accordance with an embodiment of the present invention can include an elongate shaft with a balloon disposed about a distal portion of the elongate shaft for urging core material from one or more vulnerable plaques. One or more sets of collection lumens may be disposed around and/or inside the elongated shaft with the distal ends of the collection lumens functioning as collection ports. The proximal ends of the collection lumens fluidly coupled to a suction means for extracting the core material urged from the vulnerable plaque, thrombi, debris, etc. from inside the lumen of the blood vessel. In one embodiment, a first set of one or more collection ports are disposed proximally of the balloon, and/or a second set of one or more collection ports may be disposed distally of the balloon for collecting the core material urged from the vulnerable plaque, thrombi, debris, etc.

A method in accordance with an embodiment of the present invention could include the steps of inserting the distal portion of a catheter having a balloon disposed about a distal portion of the catheter into a lumen of the blood vessel. The next step can be positioning the balloon proximate one or more vulnerable plaque, inflating the balloon using methods well known in the art, urging core material from the one or more vulnerable plaques, and providing suction means for extracting and collecting the disposed core material, thrombi, debris, etc., proximally of the balloon using a first set of one or more collection ports, and/or collecting the disposed core material, thrombi, debris, etc., distally of the balloon using a second set of one or more collection lumens.

### BRIEF DESCRIPTION OF THE DRAWINGS

FIG. 1 is a schematic view of a catheter in accordance with an embodiment of the present invention;

FIG. 2 is a view of cross-section A-A of the catheter of FIG. 1;

FIG. 3 is a view of the catheter of FIG. 1 located proximate of a plaque;

FIG. 4 is an illustration of the extraction of core material by a method of the present invention; and

FIG. 5 is an illustration of the extraction of core material by another method of the present invention.

### DETAILED DESCRIPTION OF THE INVENTION

The following detailed description should be read with reference to the drawings, in which like elements in different drawings are numbered in like fashion. In some cases, the drawings are highly diagrammatic in nature. Examples of constructions, materials, dimensions, manufacturing processes, etc., for various elements are provided for illustration purposes only. Those skilled in the art will recognize that many of the examples provided could have suitable alternatives that may be utilized without departing from the scope and spirit of the present invention.

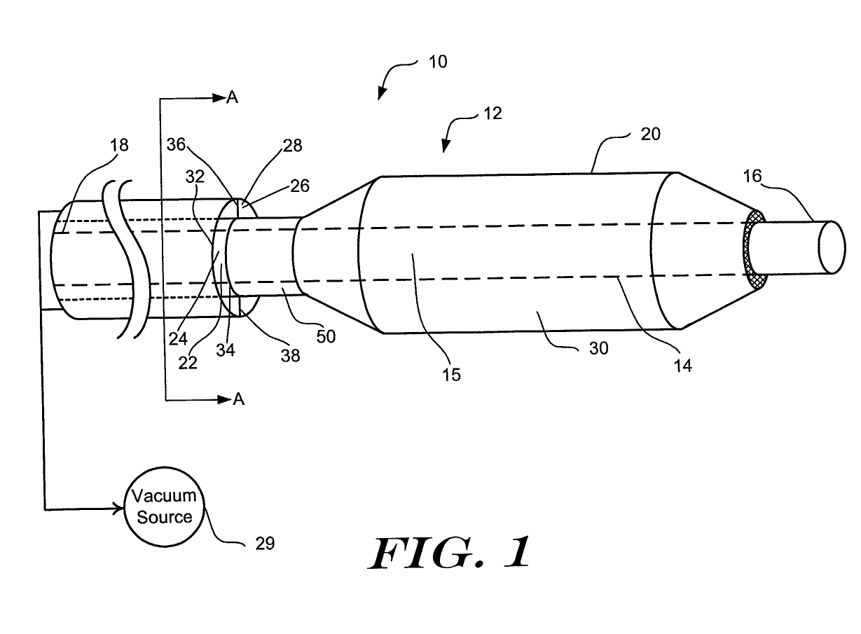

FIG. 1 is a schematic of a catheter 10 in accordance with an embodiment of the present invention. Catheter 10 may include elongated shaft 14 having a distal end 16 and a proximal end 18. An expandable member, such as balloon 20, having an expanded state and a contracted state, may be disposed about distal portion 12 of elongated shaft 14. Balloon 20 could be configured such that in the expanded state, engagement surface 30 thereof engages the inner surface of the lumen of a blood vessel and/or the one or more vulnerable plaque in the lumen of the blood vessel.

In accordance with one embodiment of the present invention, catheter 10 includes a plurality of walls 32, 34, 36, and 38 around elongated shaft 14. Said walls of catheter 10 may define a first collection lumen 22 having a first collection port 24 disposed proximally of balloon 20, and a second collection lumen 26 having a second collection port 28 also disposed proximally of balloon 20. Lumens 22 and 26 could longitudinally traverse the length of elongated shaft 14, and may be fluidly connectible to a suction means at their proximal ends. Collection lumens 22 and 26 may thus provide fluid coupling between first and second collection ports 24 and 28, respectively, and the suction means. Vacuum source 29, for example, may be used as the suction means fluidly coupled to collection ports 24 and 28 through which thrombi, debris, core material urged from the one or more vulnerable plaques, etc., could be removed from the lumen of the blood vessel.

In accordance with designs and methods of use of intravascular catheters, elongated shaft 14 may also include lumen 50 therethrough for inflating and/or deflating balloon 20, and/or guidewire lumen 15 therethrough, as is well known in the art. A fluid source may be coupled to the proximal end of lumen 50. The distal portion of lumen 50 may be in fluid communication with balloon 20 through an orifice such that lumen 50 may be used for injecting and/or removing fluids from balloon 20, as is well known in the art, for the purpose of inflating and/or deflating balloon 20. For the purposes of this disclosure, the term fluid refers to a liquid and/or a gas.

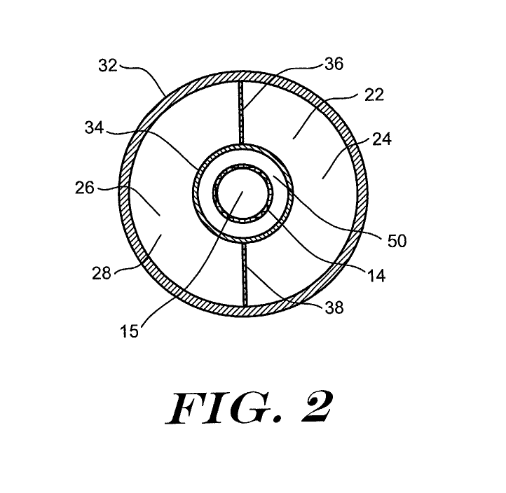

FIG. 2 is a view of cross-section A-A, proximal of balloon 20, of catheter 10 of FIG. 1. In this illustrated configuration, walls 32, 34, 36, and 38 form first and second collection lumens 22 and 26, respectively, said collection lumens providing fluid coupling between first and second collection ports 24 and 28, respectively, and suction means connected at the proximal end of said collection lumens.

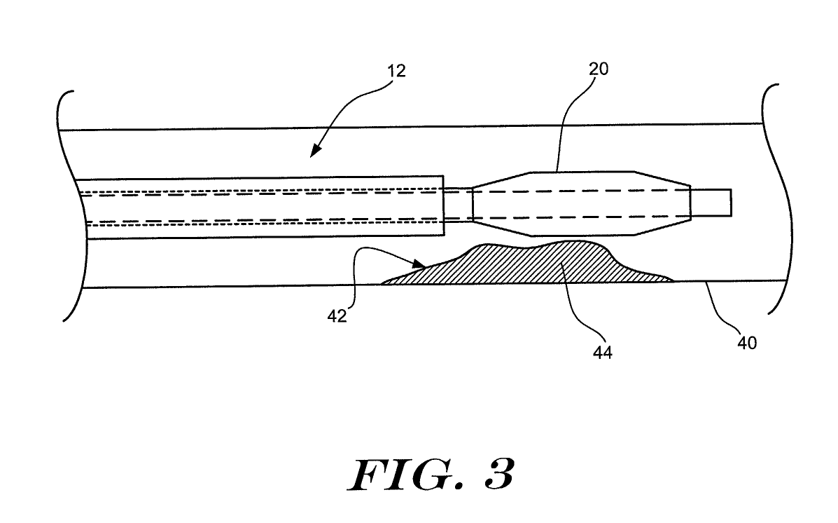

In accordance with an embodiment of the present invention, FIG. 3 shows distal portion 12 of catheter 10 of FIG. 1 as located within the lumen of blood vessel 40. Balloon 20 is shown having a deflated shape and positioned proximate of vulnerable plaque 42 within the lumen of blood vessel 40. Vulnerable plaque 42 is shown attached to the inner wall of the lumen of blood vessel 40, and containing core material 44 therein.

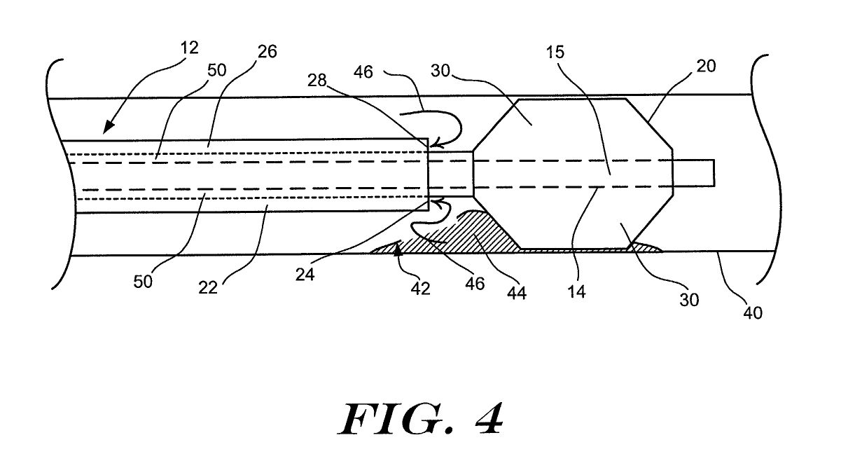

FIG. 4 is an illustration of catheter 10 of an embodiment of the present invention as it may be used for removing venerable plaque 42. Balloon 20, located about distal portion 12 of catheter 10, may be positioned proximate of vulnerable plaque 42, and then expanded to an inflated state as shown in FIG. 4. Lumen 50, which longitudinally traverses elongated shaft 14 and is in fluid communication with balloon 20, could be used for injecting and/or removing fluids at the proximal end for the purpose of inflating and/or deflating balloon 20, as is well known in the art. In one embodiment, balloon 20, when inflated, and engagement surface 30 thereof is adapted for engaging both the inner wall of the lumen of blood vessel 40 and the one or more vulnerable plaque 42. With balloon 20 thus configured, vulnerable plaque 42 may be ruptured proximal of inflated balloon 20 urging core material 44 from vulnerable plaque 42. In FIG. 4, arrows 46 illustrate the movement of urged core material 44 towards collection ports 24 and 28 proximal of balloon 20. Suction means, applied at the proximal end of collection lumens 22 and 26 and in fluid communication with respective collection ports 24 and 28, could extract core material 44 proximal of balloon 20.

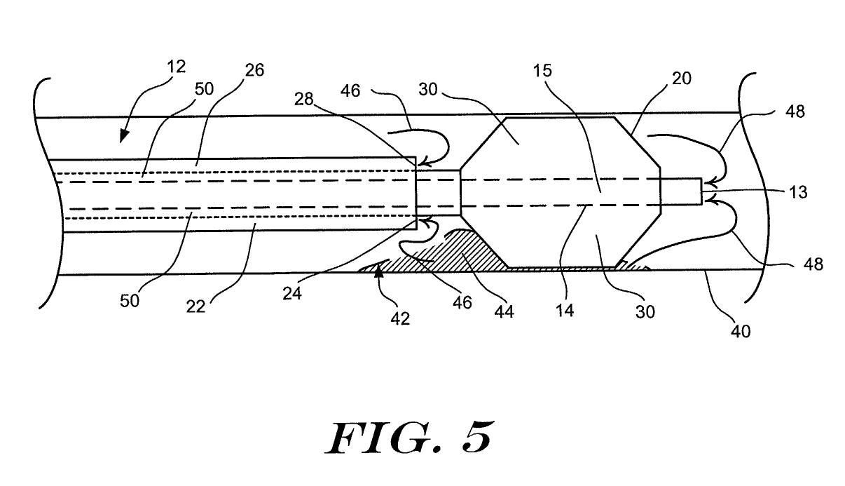

FIG. 5 is an illustration of another embodiment and method wherein lumen 15, having a proximal end and a distal end, serves dual functions as a guidewire lumen and as a third collection lumen. The distal end of lumen 15 could function as a third collection port 13 in fluid communication with a suction means fluidly coupled to the proximal end of lumen 15. In use, lumen 15 may first be used for positioning distal portion 12 of catheter 10 proximate vulnerable plaque 42. As illustrated, vulnerable plaque 42 may rupture and urge core material 44 both distally and proximally of inflated balloon 20. Arrows 48 show distal movement of urged core material 44 towards third collection port 13. Suction means applied at the proximal end of lumen 15 and in fluid communication with collection port 13 could be used for extracting core material 44 from the lumen of blood vessel 40 distal of balloon 20. As previously described, suction means applied at the proximal end of collection lumens 22 and 26 and in fluid communication with respective collection ports 24 and 28, could extract core material 44 proximal of balloon 20.

In some instances, the presence of core material 44 within blood vessel 40 may be associated with the presence of thrombi or other debris. When this is the case, thrombi, debris, etc., may also be extracted through collection ports 24 and 28 located proximal of balloon 20, and collection port 13 positioned distal of balloon 20.

Having thus described some illustrative embodiments of the present invention, those of skill in the art will readily appreciate that yet other embodiments may be made and used within the scope and spirit of the claims hereto attached. For instance, in an alternate embodiment, the dual functioning lumen 15 may be replaced by two distinctly separate lumens, one serving as a guidewire lumen and the other specifically functioning as a collection lumen. In yet another embodiment, lumen 15 might function solely as a collection lumen and encase a distinctly separate guidewire lumen therein, or vice versa.

It should be understood that this disclosure is, in many respects, only illustrative. Changes may be made in details, particularly in matters of shape, size, and arrangement of parts without exceeding the scope of the invention.

We claim:

1. A catheter for removing core material from one or more plaque deposits on an inside wall of a blood vessel, said catheter comprising:
an elongate shaft having a proximal end and a distal end;
an expandable member disposed about a portion of the elongated shaft proximate the distal end of the elongate shaft; and
a first collection lumen having a proximal end and a distal end, the distal end being disposed proximate the balloon.

2. The catheter of claim 1 further comprising a suction means fluidly coupled to the proximal end of the first collection lumen.

3. The catheter of claim 1, wherein the expandable member includes a balloon having an inflated state and a deflated state.

4. The catheter of claim 1, wherein the distal end of the first collection lumen is directed axially.

5. The catheter of claim 1, wherein the distal end of the first collection lumen is directed radially.

6. The catheter of claim 1, wherein the distal end of the first collection lumen is disposed proximal of the expandable member.

7. The catheter of claim 1, wherein the distal end of the first collection lumen is disposed distal of the expandable member.

8. The catheter of claim 1, wherein a wall of the first collection lumen is defined, at least in part, by a wall of the elongated shaft.

9. The catheter of claim 1 further comprising a second collection lumen having a proximal end and a distal end, the distal end being disposed proximate the expandable member.

10. The catheter of claim 9 further comprising a vacuum means fluidly coupled to
the proximal end of the first collection lumen; and
the proximal end of the second collection lumen.

11. The catheter of claim 9, wherein the distal end of the second collection lumen is directed axially.

12. The catheter of claim 9, wherein the distal end of the second collection lumen is directed radially.

13. The catheter of claim 9, wherein the distal end of the second collection lumen is disposed proximal of the expandable member.

14. The catheter of claim 9, wherein the distal end of the second collection lumen is disposed distal of the expandable member.

15. The catheter of claim 9, wherein a wall of the second collection lumen is defined, at least in part, by a wall of the elongated shaft.

16. The catheter of claim 9, wherein a wall of the second collection lumen is defined, at least in part, by a wall of the first collection lumen.

17. The catheter of claim 9, wherein one of the first or second collection lumen is a guidewire lumen.

18. A method for removing core material from one or more plaque deposits on an inside wall of a blood vessel, said method comprising the steps of:
providing a catheter having an elongate shaft with a proximal end and a distal end, an inflatable balloon disposed about a portion of the elongated shaft proximate the distal end of the elongate shaft, one or more collection lumens each having a proximal end fluidly coupled to a suction means and a distal end disposed proximate of the balloon;
inserting the distal end of the catheter into a lumen of the blood vessel;
positioning the balloon proximate a plaque deposit;
inflating the balloon to engage the inner surface of the blood vessel and the one or more plaque deposits and rupturing the plaque deposits in one or more locations urging the core material from the plaque deposits;
collecting the urged core material from a location proximate of the balloon using the distal end of the one or more collection lumens; and
extracting the urged core material from the lumen of the blood vessel with suction means fluidly coupled to the proximal ends of the one or more collection lumens.
